(19) United States
(12) Patent Application Publication
(10) Pub. No.: US 2025/0325244 A1
(43) Pub. Date: Oct. 23, 2025

(54) COMPUTER-IMPLEMENTED METHOD FOR OPERATING A MEDICAL IMAGING DEVICE, MEDICAL IMAGING DEVICE, COMPUTER PROGRAM, AND ELECTRONICALLY READABLE STORAGE MEDIUM

(71) Applicants: Siemens Healthineers AG, Forchheim (DE); Herlev and Gentofte Hospital, Herlev (DK)

(72) Inventors: Oliver TAUBMANN, Weilersbach (DE); Michael SUEHLING, Erlangen (DE); Adam William Augustinus PAASKE, Kolding (DK); Christoph Felix MUELLER, Copenhagen (DK)

(73) Assignees: Siemens Healthineers AG, Forchheim (DE); Herlev and Gentofte Hospital, Herlev (DK)

(21) Appl. No.: 19/181,477

(22) Filed: Apr. 17, 2025

(30) Foreign Application Priority Data
Apr. 18, 2024 (EP) 24170983.1

(51) Int. Cl. A61B 6/00; A61B 6/03; A61B 6/46

(52) U.S. Cl. CPC A61B 6/545; A61B 6/03; A61B 6/463; A61B 6/488; A61B 6/5247; A61B 6/5264; A61B 6/5294

(57) ABSTRACT

A method comprises: retrieving, from a storage, a source data set chosen from at least one prior imaging result data set, each prior imaging result data set including a prior image showing at least part of an acquisition region and annotation information, wherein the annotation information includes location information describing a location of a respective feature of interest in a second coordinate system of the prior image; registering the prior image and the preparation image to obtain registration information including a transformation from the second coordinate system to the first coordinate system; compiling an output image including the preparation image and at least one visual representation, which is positioned correctly according to the location information of the source data set for at least one direction, of all features of interest within a display region comprising the acquisition region; and outputting the output image at a display device.

### CROSS-REFERENCE TO RELATED APPLICATION(S)

The present application claims priority under 35 U.S.C. § 119 to European Patent Application No. 24170983.1, filed Apr. 18, 2024, the entire contents of which is incorporated herein by reference.

### FIELD

One or more example embodiments of the present invention concern a computer-implemented method for operating a medical imaging device, wherein, for preparing an examination scan of a subject, a preparation image of an acquisition region is acquired, the preparation image being defined with respect to a first coordinate system and used to define at least one parameter for acquiring an examination image. One or more example embodiments of the present invention further concern a medical imaging device, a computer program, and non-transitory electronically readable storage medium.

### BACKGROUND

In medical imaging, it is known to acquire preparation images. Based on such a preparation image, acquisition parameters for the acquisition of at least one examination image may be defined. For example, the preparation image may provide an overview over the anatomy of the subject, in particular a patient, to choose the field of view such that it comprises features of interest, for example certain anatomical structures like organs or lesions. In computed tomography (CT), a topogram is often required as preparation image. A topogram is a two-dimensional x-ray projection image acquired with the computed tomography device and used by the operator to (manually and/or automatically) define the scan range, in particular in the axial direction, and/or the field of view for reconstruction. The topogram may also be used to identify (manually and/or automatically) potential issues that may impede diagnostic image quality, for example the presence of metal objects. Appropriate countermeasures may be taken. In magnetic resonance imaging (MRI), localizers can be acquired as preparation images.

Medical imaging is routinely used to diagnose diseases, such that they can be treated, and to monitor the progress of therapy. Often, so-called follow-up examination scans of subjects, in particular patients, are performed. These examination scans may, for example, serve to monitor the progression or healing of medical conditions. For example, a patient diagnosed with cancer may undergo CT scans several times over the course of the therapy to monitor the response of the disease. In order to plan an examination scan, it is important to know the location and extent of the internal feature of interest to be examined.

In the state of the art, to ensure that all relevant features are inside the planned scan range and/or field of view and are reconstructed at sufficient resolution, the user relies on communication with a referring physician and/or radiologist to learn of any subject-specific requirements and/or needs to manually check prior scans/findings in a different system, for example a picture archiving and communication system (PACS) or an electronic patient record.

This solution is prone to errors, since there may be miscommunication (or even a lack of communication regarding a specific aspect) between the radiologist/clinician and the user of the medical imaging device (the radiographer), and/or the user does not have the capacity, in particular regarding time and expertise, to manually check prior scans or check them diligently enough. In this case, an examination scan may have to be repeated. Even if no errors are made, the need for additional communication or manual search for and checking of prior scans introduces inefficiencies in the workflow.

### SUMMARY

It is an object of embodiments of the present invention to provide improved support to users of medical imaging devices regarding the planning of the acquisition of an examination image, in particular such that at least one feature of interest is imaged.

At least this object is achieved by providing a computer-implemented method, a medical imaging device, a computer program and non-transitory electronically readable storage medium as claimed.

According to an embodiment of the present invention, a method as initially described comprises the steps of:
retrieving, from a storage comprising at least one prior imaging result data set of the subject, a source data set chosen from the at least one prior imaging result data set, each prior imaging result data set comprising a prior image showing at least a part of the acquisition region and annotation information indicating at least one feature of interest of the subject, wherein the annotation information comprises a location information describing a location of a respective feature of interest in a second coordinate system of the prior image,
registering the prior image of the source data set and the preparation image to obtain registration information comprising a transformation from the second coordinate system to the first coordinate system,
compiling an output image comprising the preparation image and at least one visual representation, which is positioned correctly according to the location information of the source data set for at least one direction, of all features of interest within a display region comprising the acquisition region, and
outputting the output image at a display device.

It is hence proposed to automatically identify suitable prior images, register them to the preparation image of the current examination scan, and subsequently display, in particular in a user interface showing the preparation image, any relevant features of interest, for example prior findings, determined in prior scans of the subject. As a result of the registration, the at least one feature of interest is displayed at the correct location regarding the preparation image. In particular, visual representations of the features of interest may be overlaid onto the preparation image or positioned relative to the preparation image in the display region at the correct position in at least one direction, since the transformation can be used to transfer the location information from the second coordinate system to the first coordinate system. In other words, the output image is generated for the display region in the second coordinate system using the preparation image, the annotation information of the source dataset and the transformation, which is applied to the location information. Key components of the method hence comprise registration of a prior scan to the preparation image of the current examination scan and the subsequent display of prior findings with the preparation image based on the registration result. It is applied to stand-alone examination scans, meaning the prior imaging result data sets relate to prior scans a longer time ago, for example at least one month ago.

The output image may be part of a user interface, wherein the user can choose and/or adapt and/or confirm acquisition parameters, in particular acquisition parameters defining the field of view for acquisition and/or reconstruction of the examination image. The information provided in the output image can hence be used to choose improved acquisition parameters, in particular ensure that the at least one feature of interest is in the field of view and/or depicted in sufficiently high quality.

The herein presented method has advantages regarding multiple aspects of the method. Prefetching of a suitable prior image and its registration to the preparation image circumvent the need for manual search and visual comparison by the user. By being able to automatically display the features of interest, in particular prior findings, directly in the preparation image view, no additional manual efforts are required, and the likelihood of mistakes is reduced. Hence, medical imaging devices become smarter and more user-friendly. In particular, follow-up examinations (which account for a very large portion of all examination scans), for example regarding CT scans of oncology patients, may be carried out more quickly and with a reduced probability for errors.

Hence, preferably, the examination scan is a follow-up examination scan, wherein the at least one prior imaging result data set relates to a previous examination scan and/or is a baseline scan and/or wherein the examination scan is performed for comparison with the at least one prior imaging result data set. For example, the prior image and annotation information may relate to a prior scan, which has been acquired a certain time interval before the current scan, for example at least one month ago. The examination scan may be part of a series of scans, in particular at regular or irregular intervals, to monitor how well a treatment works, a wound heals and/or how a medical condition evolves. In particular, the examination image or results of an analysis of the examination image may be compared to the prior image and/or the annotation information or other previous analysis results. The source data set and/or further prior imaging result data sets may hence also be used regarding analysis of the examination image, in particular regarding at least one of the at least one feature of interest. For example, the growth or other evolution of a lesion can be evaluated.

The method may be applied to any imaging modality and/or process using a preparation image, for example localizers of medical resonance imaging. Preferably, however, the medical imaging device is a computed tomography device, and the preparation image is a topogram. Computed tomography relies on x-rays, such that the method may advantageously also result in less radiation to be applied to the subject, in particular patient.

Regarding CT, the at least one direction may comprise at least an axial direction, which corresponds to the superior-inferior direction of the patient. That is, relative positioning of the subject regarding the acquisition of the examination image is most important along the rotational axis of the acquisition arrangement (x-ray source and x-ray detector) of the medical imaging device, which also is the axial direction of the gantry. It usually also corresponds to the longitudinal direction of the patient table. By providing information regarding the axial position of the at least one feature of interest, the radiation may be directed and confined to the correct and required part of the body.

While, in principle, the source data set may be chosen manually, it is preferred to provide automatic selection of the source data set. Hence, preferably, the source data set is chosen from the multiple prior imaging result data sets using at least one selection function, wherein the selection function evaluates at least one selection criterion. Here, at least one of the at least one selection criterion may be chosen from the group comprising
the acquisition date of the prior imaging result data set, wherein more recent prior imaging result data sets are preferred,
the body region the prior imaging result data set relates to, wherein prior imaging result data sets relating to the same body region as the examination scan are preferred,
the modality the prior imaging result data set has been acquired with, wherein prior imaging result data sets being acquired with the same (or at least a similar) modality as the one used for the examination scan are preferred,
motion state information associated with the prior image, wherein prior images having a similar motion state to the preparation image are preferred, and
acquisition protocol parameters, wherein prior imaging result data sets using acquisition protocols similar to that of the acquisition scan are preferred.

Selection and automated provision of the source data set at the medical imaging device may take multiple selection criteria into account. For example, advanced selection functions for identifying suitable prior scans, in particular employing machine learning/artificial intelligence, which have already been proposed in the state-of-the-art in other contexts, may also be applied in the present invention. Hence, a trained function may be used as the selection function.

Generally, prior images being acquired with the same modality and showing the same body region may be preferred, since registration is facilitated. In particular, furthermore, similar acquisition protocols also simplify registration. For example, similarity scores may be determined and used. In embodiments, motion state information may be associated with the prior image, describing at least one motion state, in which the prior image or a respective sub-image has been acquired, the motion state relating to breathing motion and/or heart motion. Here, similar motion states are preferred, further simplifying the registration process. However, a main and important selection criterion is the acquisition date, in particular regarding features of interest, for example in regard of monitoring their evolution. If multiple selection criteria are used, they may be weighted differently, for example assigning high weight to the acquisition date and the body region and lower weights to other selection area.

However, in a simple heuristic approach sufficient for many applications, the prior imaging result data set relating to the latest scan of the body region under examination, in particular the acquisition region or the display region, may be chosen as the source data set.

In embodiments, the at least one prior imaging result data set may be retrieved from an electronic patient record (often also electronic health record-EHR). In preferred embodiments, all prior imaging result data sets in the electronic patient record may be stored using a common second coordinate system. In such a case, prior images may be used for registration irrespective of acquisition date and up-to-dateness. For example, a prior imaging result data set for use as the source data set can be compiled comprising a prior image which is optimal for registration and the most current annotation information regarding the body region subject to examination.

The source data said can automatically be prefetched, that is retrieved, from a storage location external to the medical imaging device, for example from a picture archiving and communication system (PACS).

In embodiments, the prior image may be a preparation image of a prior examination scan using the same modality, simplifying the registration. This may hence provide an incentive to also store preparation scans in long-term storages like PACS. For example, both the preparation image and the prior image may be topograms, allowing easy and robust 2D-2D registration.

In other preferred embodiments, the preparation image is two-dimensional, and the prior image is at least three-dimensional, such that registration is performed as 2D-3D-registration. For example, a topogram may be registered to a three-dimensional prior image, in particular a prior computed tomography volume and/or stack of sectional images/slice images. 2D-3D-registration algorithms and functions have already been proposed in the state of the art. In preferred embodiments, the 2D-3D-registration comprises at least one of
landmark-based registration, and
minimizing a difference metric between preparation image and a two-dimensional reference image determined, in particular by forward projection, from the prior image, in particular based on an assumed acquisition geometry of the preparation image in the second coordinate system.

For example, known landmarks of corresponding anatomical structures/locations may be automatically detected in both the preparation image and the prior image. Thereafter, as the transformation, a suitable geometric transformation (for example a homography) that maps the 3D landmark positions to their respective 2D landmark positions, in particular projected positions in the case of a topogram, may be determined. Additionally or alternatively, a difference metric between the preparation image and reference image can be minimized. For example in the case of a topogram, the reference image may be a virtual (simulated/computed) X-ray projection image (DRR, “digitally reconstructed radiograph”) from the 3D prior image, projected according to an estimate of the projection geometry (as acquisition geometry) that is refined during the optimization process. Generally speaking, the assumed acquisition geometry may also be optimized during registration, in particular the optimization process.

In particular regarding body regions which are subject to physiological motion, in particular breathing motion and/or heart motion, like abdomen or thorax, the motion state may also be taken into account. As discussed above, motion state information may be associated with the prior image, describing at least one motion state, in which the prior image or a respective sub-image has been acquired, the motion state relating to breathing motion and/or heart motion. A suitable three-dimensional prior image can be selected (as already discussed above) and/or a suitable three-dimensional sub-image of the four-dimensional prior image can be chosen for registration based on the motion state information.

However, other effects may also impede registration quality. For example, the size and/or position of inner organs may change over time, preventing perfect registration, which, however, is not required in this application. In particular, it may be sufficient to use rigid registration.

In preferred embodiments, the preparation image may be a two-dimensional projection image, in particular a topogram, and the annotation information may comprise a three-dimensional location information, wherein, during compilation of the output image, the three-dimensional location information is forward projected into the projection geometry of the preparation image according to the transformation to facilitate correct positioning of the respective feature in the output image. Hence, any (relevant) features, in particular findings, of the prior scan of the source data set may be projected using the projection geometry of the preparation image to be displayed in the anatomically corresponding position in the preparation image view, that is, the output image.

In especially preferred embodiments, if a feature lies outside the acquisition region according to the transformation, a warning may be output to a user and/or the feature may be displayed at the correct position according to the location information outside the preparation image in the display region. For example, if a feature of interest lies outside the acquisition region, a notice can be displayed to the user, informing him of this shortcoming. For example, the user may then consider acquiring another preparation image with an adapted field of view, for example, in the case of topogram, an adapted axial scan range, wherein the respective feature of interest is comprised in the new, adapted field of view. To provide further information, in particular regarding adaptation of the field of view for a further preparation image and/or regarding manual and/or automatic adjustment of the field of view of the examination image, in preferred embodiments, wherein the display region is larger than the acquisition region or can be suitably chosen, the location of the feature of interest outside the acquisition region, at least in the axial direction, can be correctly displayed in the display region, in particular to scale. Hence, the user can easily determine required adaptations of the field of view.

Preferably, at least one acquisition parameter for the examination image, which describes the field of view of the examination image, in particular an axial examination range, is automatically adapted such that the feature lying outside the acquisition region is included in the field of view of the acquisition image. Hence, for example, proposals for adapted acquisition parameters may be automatically determined, in particular such that the feature of interest is included in the field of view of the acquisition image, and may be presented to the user for confirmation. In this manner, further support for the user is provided.

It is noted that acquisition parameters of the examination image, in particular as proposals to be confirmed by the user, may also be determined automatically for other use cases, for example regarding desired quality of depiction of the feature of interest in the examination image. Preferably, such a proposal is at least partly also based on annotation information, for example the type of feature.

Preferably, an expected location error in at least one direction, in particular the superior-inferior and/or axial direction, due to breathing and/or registration is determined and visualized in the output image by at least one visualization element. In this manner, potential misalignment due to organ movement and/or registration errors can be accounted for. For example, a margin of error could be added to the visual representation of the feature of interest, in particular its location in at least one direction. If, for example, an axial dimension of the feature of interest, for example by marking an axial range, is indicated in the output image, extensions to this visual representation/display element of the visual representation may be added. For example, in the thorax or abdomen area, typical values for superior-inferior displacement due to breathing, which is the main contributor of misalignment, may be used. Expected breathing motion may be taken from a database of empirically determined values, in particular associated with body regions and/or anatomical structures, and/or may be determined based on patient-specific prior information, which may, for example, be comprised in the source data set and/or at least one other prior imaging result data set, for example associated with a four-dimensional prior image. Such 4D scans are, for example, known from radiation treatments. Additional information regarding potential misalignment due to breathing allow to choose the field of view of the examination image to reliably acquire image data from the corresponding feature of interest. If, in less preferred embodiments, registration errors are taken into account, these could be provided by used registration functions.

Features of interest, in particular prior findings, may be provided in prior imaging result data sets in different forms. For example, the annotation information may comprise at least one chosen from the group of
DICOM meta information,
a reference to a partial image of the or a prior image, in particular to a slice,
at least one extension of the feature, in particular a lesion diameter,
a textual entry describing the position of the feature in at least one of the at least one direction, and
an, in particular three-dimensional, segmentation result of the feature.

In concrete embodiments, often used simple annotation information, like diameters of a target lesion in a single slice or sectional image or references to slice numbers, can already be used to roughly determine the location of the feature of interest in at least one direction, for example the axial direction (which usually also is the stacking direction of sectional/slice images). Furthermore, textual information, for example diagnosis comments, may also be analysed as location information. In preferred, ideal cases, the annotation information may comprise a full three-dimensional, automatic or semi-automatic segmentation of the feature of interest. Annotation information may be stored in prior images as DICOM meta information, using well-known standard.

Embodiments of the present invention further concern a medical imaging device, in particular computed tomography device, for an examination scan of a subject, comprising an acquisition arrangement, a display device and a control device, wherein the control device comprises:
an acquisition unit for controlling the acquisition arrangement to acquire a preparation image of an acquisition region, the preparation image being defined with respect to a first coordinate system and used to define at least one parameter for acquiring an examination image, and the examination image,
a storage interface for retrieving, from a storage comprising at least one prior imaging result data set of the subject, a source data set chosen from the at least one prior imaging result data set, each prior imaging result data set comprising a prior image showing at least a part of the acquisition region and annotation information indicating at least one feature of interest of the patient, wherein the annotation information comprises a location information describing a location of a respective feature of interest in a second coordinate system of the prior image,
a registration unit for registering the prior image of the source data set and the preparation image to obtain registration information comprising a transformation from the second coordinate system to the first coordinate system,
a compilation unit for compiling an output image comprising the preparation image and at least one visual representation, which is positioned correctly according to the location information of the source data set for at least one direction, of all features of interest within a display region comprising the acquisition region, and
an output unit for outputting the output image at the display device.

The control device may comprise at least one processor and at least one storage device or storage means. It may be connected, in particular via a communication link, to the storage, wherein the prior imaging result data sets are stored. Functional units may be implemented by software and/or hardware to perform steps of a method according to embodiments of the present invention. Acquisition units for controlling the acquisition arrangement to acquire image data are already known. A reconstruction unit may be associated with the acquisition unit or integrated into the acquisition unit for reconstructing images from raw data. In addition to the registration unit, the compilation unit and the output unit, which may generally be responsible for providing a user interface, additional functional units may be provided to perform steps of additional embodiments of the present invention. For example, the control device may also comprise a selection unit for selecting the source data set from multiple prior imaging result data sets.

A computer program according to embodiments of the present invention comprise program means such that, when the computer program is executed on a control device of a medical imaging device, the control device is caused to execute the steps of a method according to embodiments of the present invention. The computer program may be stored on a non-transitory electronically readable storage medium according to embodiments of the present invention, which thus comprises control information comprising at least one computer program according to embodiments of the present invention, such that, when the storage medium is used in a control device of a medical imaging device, the control device is configured to perform a method according to embodiments of the present invention. The storage medium may preferably be a non-transient storage medium, for example a CD-ROM.

### BRIEF DESCRIPTION OF THE DRAWINGS

Other objects and features of the present invention will become apparent from the following detailed description considered in conjunction with the accompanying drawings. The drawings, however, are only principal sketches designed solely for the purpose of illustration and do not limit the present invention. The drawings show:

FIG. 1 a flowchart of an embodiment of a method according to embodiments of the present invention,

FIG. 2 schematically a first output image,

FIG. 3 schematically a second output image,

FIG. 4 a medical imaging device according to embodiments of the present invention, and

FIG. 5 the functional structure of the control device of the medical imaging device of FIG. 4.

### DETAILED DESCRIPTION

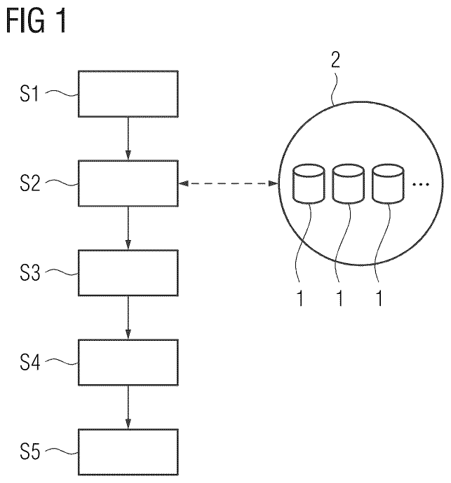

FIG. 1 shows a flowchart of a general embodiment of a method according to the present invention. The embodiment exemplarily relates to an examination scan of a patient, performed with a computed tomography device as medical imaging device. Generally, in the examination scan, in a step S1, a topogram covering an axial region of the patient (that is, a body region in the superior-inferior direction, which corresponds to the axial direction of a gantry of the computed tomography device and hence the direction of the rotational axis of the acquisition arrangement) is acquired as a preparation image. The topogram is a two-dimensional projection image defined in a first coordinate system used by the medical imaging device. As other preparation images, for example localizers in MRI, the topogram is used to defined acquisition parameters for the main scan to acquire an examination image (as a computed tomography image reconstructed from multiple projections) of the patient. In particular, the topogram is used to ensure that all regions of interest are included in the field of view of the examination image. Hence, the topogram (or generally, the preparation image) is usually shown in a user interface on a display device of or associated with the medical imaging device. In the method described here, this view is extended by adding visual representations of features of interest, in particular prior findings.

Therefore, in a step S2, a source data set is selected from multiple prior imaging result data sets 1 in a storage 2 and retrieved from the storage 2. Each prior imaging result data set 1 comprises a prior image and annotation information regarding at least one feature of interest and its location with regard to a second coordinate system, in which the prior image is defined. For example, the prior image can be stored in a DICOM format, and the annotation information may be, at least partly, included as meta data. The location information included in the annotation information may be a rough description of the location of the feature of interest in at least one direction, for example a slice number in a slice stack or a textual description, but is preferably more precise. For example, the annotation information may comprise a, preferably 3D, segmentation result and/or a measurement result regarding at least one extension of the feature. Features of interest are, preferably, prior findings, for example lesions.

In this example, the examination scan is a follow-up examination scan, for example for monitoring the progress of a treatment/a therapy and/or the development of a disease, for example a tumor or other medical condition. In this sense, the examination scan may be understood as a stand-alone scan, meaning the prior scans described by the prior imaging result data sets 1 were acquired with a temporal distance, for example at least one month ago.

For selecting a suitable prior imaging result data set 1 as source data set, selection criteria may be evaluated, in particular in a weighed manner. Such selection criteria may comprise the date of acquisition of the prior image, the body region examined, the acquisition protocol used, the state of motion shown in the prior image regarding breathing and/or heart motion, and the imaging modality used. The more similar the prior image is to the preparation image, the easier the registration, however, proximity in time is also important, in particular regarding planned comparisons of the examination image and/or its annotations with prior scan results.

In an easily implementable approach, that is likely to suffice in most scenarios, the prior imaging result data set 1 relating to the latest scan of the same body region in the patient is selected as the source data set. In other approaches, however, more complex selection functions, in particular trained selection functions, may also be employed in step S2.

The storage 2 may be a PACS. In embodiments, the prior imaging result data sets 1 may be stored in an electronic patient record of the patient. They may preferably all use the same second coordinate system, allowing to compile and select prior imaging result data sets 1 comprising a prior image optimal for registration to the preparation image and the latest annotation information regarding relevant features of interest.

In a step S3, the prior image of the source data set and the preparation image, in this case the topogram, are co-registered to determine a transformation from the second coordinate system to the first coordinate system. In other words, the coordinate systems need to be aligned to establish spatial correspondence such that features of interest identified in the prior scan can be displayed at the corresponding anatomical position in the preparation image.

In some embodiments, the preparation image may also be a topogram, preferably using the same projection plane (for example a coronal plane). The prior image may even have been acquired with the same or a comparable medical imaging device, for example in a prior scan of a series of examination scans relating to a certain feature of interest. In such a case, 2D-2D registration may be performed in step S3.

However, in many cases, the prior image may be a three-dimensional image, for example a reconstructed computed tomography or magnetic resonance volume and/or slice stack. 2D-3D registration is performed. Exemplary concrete approaches comprise landmark-based methods and approaches trying to minimize a difference metric regarding the respective image information. In the topogram embodiment discussed here, a projection geometry may be assumed, and a reference image may be determined by forward projecting from the three-dimensional prior image. Such a forward projected reference image is also known as DRR (digitally reconstructed radiograph) and may be determined by simulation or direct computation. A difference metric between the reference image and the preparation image is minimized, also refining the acquisition geometry in the optimization process.

It is noted that perfect registration may not be possible in every case. While, when the prior image is four-dimensional covering a full breathing and/or heart cycle, a suitable sub-image showing the same motion state may be selected for registration to reduce deviations due to breathing and/or heart motion, such motion, in particular breathing motion, may also be taken into account when preparing an output image for display, as further discussed below. Perfect registration, however, is no prerequisite for the support option for the user provided here.

In a step S4, an output image is determined from the preparation image, the annotation information and the transformation. The output image is output in step S5 to a user as part of a user interface. Given the registration performed in step S3, any features of interest annotated/detected in the prior scan of the source data set can be added to the output image, in particular, for the topogram, projected with the same projection geometry, to be displayed in the anatomically corresponding position in the display region covered by the output image.

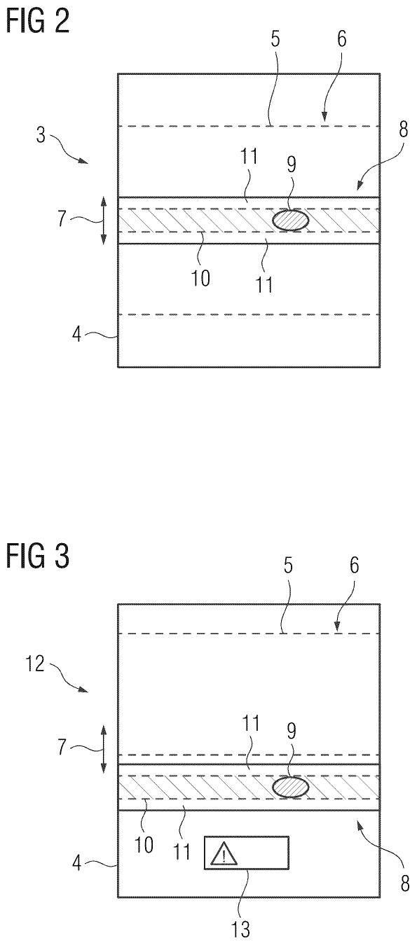

FIG. 2 shows a first example of a potential visualization. The output image 3 covers a display region 4 which is larger than, but comprises the acquisition region 5 of the included preparation image 6 in one direction, in this case the axial direction 7 (corresponding to the superior-inferior direction of the patient). In this case, the feature of interest is located within the acquisition region 5, such that its visual representation 8 comprising multiple representation elements is overlaid over the preparation image 6. The representation elements comprise the feature of interest 9 itself and an indicator 10 of its axial dimension. Furthermore, a visualization element 11 indicates a safety margin in axial direction 7. In this case the safety margin is chosen to account for potential misalignments due to breathing motion, which is often relevant in the thorax and abdomen region. Expected breathing motion amplitudes may be taken from a database of empirically determined values, in particular associated with body regions and/or anatomical structures, and/or may be determined as a patient-specific estimate based on patient-specific prior information, which may, for example, be comprised in the source data set and/or at least one other prior imaging result data set. Other possible errors may be included, for example, registration errors provided by a registration function performing the registration in step S3.

The visual representation 8 allows a user to select appropriate acquisition parameters for the examination image, in particular regarding its imaged and/or reconstructed field of view. Optionally, at least a part of such acquisition parameters may be automatically determined and confirmed by the user. For example, a proposed field of view of the examination image may additionally be shown in the user interface, in particular in the output image 3.

FIG. 3 shows an example of an output image 12, in which the feature of interest 9 lies outside of the acquisition region 5 of the topogram, but inside the (here correspondingly larger chosen) display region 4. As a warning 13, a notice is displayed that informs the user about this circumstance. The user may then consider acquiring another topogram to include that feature of interest 9 in the acquisition region. However, the visual representation 8 of the feature of interest 9 is still shown at the correct position in the display region 4, in particular also indicating the axial range (indicator 10) occupied by the feature of interest 9 and the safety margins (visualization elements 11), to facilitate visual/manual adjustment of the acquisition region 5 and/or the field of view of the examination image. In particular, an additional warning or notice can inform the user whether the feature of interest 9 is reliably included in the field of view of the examination image according to the current acquisition parameters.

In later steps not shown here, final acquisition parameters for the main scan of the examination image may be chosen/confirmed using the user interface, and the examination image may be acquired and analyzed, in particular regarding the feature of interest 9.

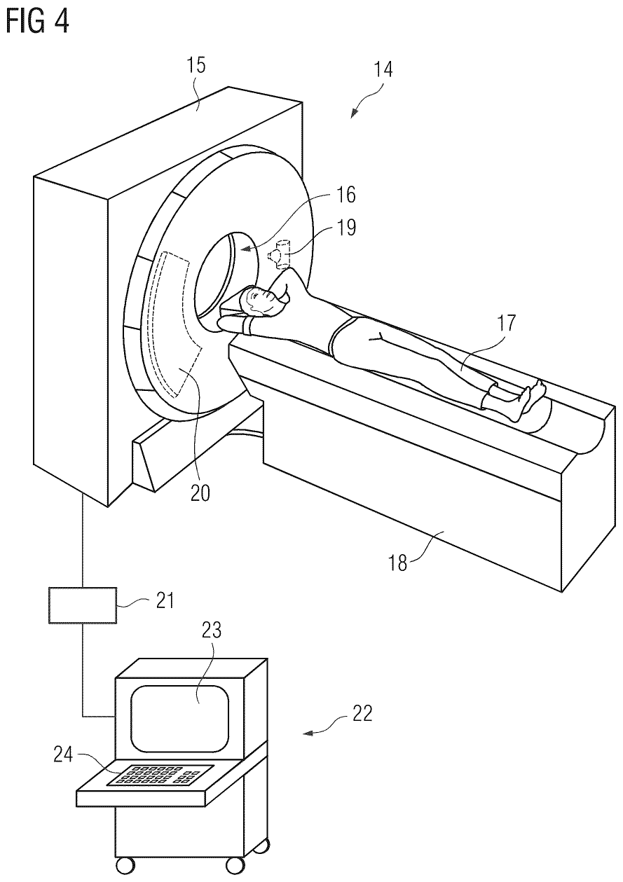

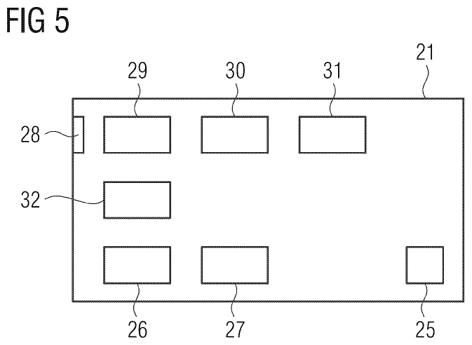

FIG. 4 shows a principle drawing of a medical imaging device 14 according to embodiments of the present invention, in this case a computed tomography device. The medical imaging device 14 comprises a gantry 15 having a patient opening 16, into which a patient 17 can be introduced using patient table 18. In the gantry 15, a rotatable acquisition arrangement comprising an x-ray source 19 and an x-ray detector 20 is placed.

Operation of the medical imaging device 14 is controlled by a control device 21, which is also configured to execute a method as described regarding FIG. 1. The control device 21 is connected to a user interface device 22, comprising a display device 23 and an input device 24.

FIG. 5 shows the functional structure of the control device 21. The control device 21 comprises a storage device or storage means 25 for storing information, for example the preparation image 6, the retrieved source data set and the like. An acquisition unit 26 controls the acquisition arrangement to acquire image data, in particular also the preparation image 6 in step S1 and the acquisition of the examination image later on. A reconstruction unit 27 may be associated with the acquisition unit 26 to reconstruct three-dimensional images from acquire two-dimensional projections, as in principle known in the art.

Via a storage interface 28, the control unit is connected to the storage 2 for retrieving the source data set selected from the prior imaging result data sets 1 according to step S2 by a selection unit 29. The control device further comprises a registration unit 30 for determining the transformation according to step S3 and a compilation unit 31 for determining the output image 3, 12 according to step S4.

An output unit 32, which generally provides the user interface outputs the output image 3, 12 on the display device 23.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for operating a medical imaging device, wherein, for preparing an examination scan of a subject, a preparation image of an acquisition region is acquired, the preparation image being defined with respect to a first coordinate system and used to define at least one parameter for acquiring an examination image, the computer-implemented method comprising:
retrieving, from a storage including at least one prior imaging result data set of the subject, a source data set chosen from the at least one prior imaging result data set, each of the at least one prior imaging result data set including a prior image showing at least a part of the acquisition region and annotation information indicating at least one feature of interest of the subject, wherein the annotation information includes location information describing a location of a respective feature of interest in a second coordinate system of the prior image;
registering the prior image and the preparation image to obtain registration information including a transformation from the second coordinate system to the first coordinate system;
compiling an output image including the preparation image and at least one visual representation, which is positioned correctly according to the location information for at least one direction, of all features of interest within a display region including the acquisition region; and
outputting the output image at a display device.

2. The computer-implemented method according to claim 1, wherein at least one of
the examination scan is a follow-up examination scan,
the at least one prior imaging result data set relates to at least one of a previous examination scan or is a baseline scan, or
the examination scan is performed for comparison with the at least one prior imaging result data set.

3. The computer-implemented method according to claim 1, wherein the medical imaging device is a computed tomography device and the preparation image is a topogram.

4. The computer-implemented method according to claim 1, wherein
The at least one prior imaging result data set includes multiple prior imaging result data sets,
the source data set is chosen from the multiple prior imaging result data sets using at least one selection function, and
the at least one selection function is configured to evaluate at least one selection criterion.

5. The computer-implemented method according to claim 4, wherein at least one of the at least one selection criterion includes at least one of
an acquisition date of a prior imaging result data set, wherein newer prior imaging result data sets are preferred,
a body region to which a prior imaging result data set relates, wherein prior imaging result data sets relating to a same body region as the examination scan are preferred,
a modality with which a prior imaging result data set has been acquired, wherein prior imaging result data sets acquired with a same modality as that used for the examination scan are preferred,
motion state information associated with the prior image, wherein prior images having a similar motion state to the preparation image are preferred, or
acquisition protocol parameters, wherein prior imaging result data sets using acquisition protocols similar to that of an acquisition scan are preferred.

6. The computer-implemented method according to claim 1, wherein the preparation image is two-dimensional and the prior image is at least three-dimensional, such that registration is performed as a 2D-3D-registration.

7. The computer-implemented method according to claim 6, wherein the 2D-3D-registration comprises at least one of landmark-based registration, or
minimizing a difference metric between the preparation image and a two-dimensional reference image.

8. The computer-implemented method according to claim 1, wherein
the preparation image is a two-dimensional projection image and the annotation information includes a three-dimensional location information, and
during compiling of the output image, the three-dimensional location information is forward projected into projection geometry of the preparation image according to the transformation to facilitate correct positioning of a respective feature in the output image.

9. The computer-implemented method according to claim 1, wherein, in response to a feature being outside the acquisition region according to the transformation, the computer-implemented method comprises at least one of
outputting a warning to a user, or
displaying the feature at a correct position according to the location information outside the preparation image in the display region.

10. The computer-implemented method according to claim 9, wherein at least one acquisition parameter for the examination image, which describes a field of view of the examination image, is adapted such that the feature outside the acquisition region is included in the field of view.

11. The computer-implemented method according to claim 1, wherein an expected location error in at least one direction due to at least one of breathing or registration is determined and visualized in the output image by at least one visualization element.

12. The computer-implemented method according to claim 1, wherein the annotation information comprises at least one of DICOM meta information,
a reference to a prior image or a partial image of the prior image,
at least one extension of a feature,
a textual entry describing a position of the feature in at least one of the at least one direction, or
a segmentation result of the feature.

13. A medical imaging device for an examination scan of a subject, the medical imaging device comprising:
an acquisition arrangement;
a display device; and
a control device, wherein the control device includes
an acquisition unit configured to control the acquisition arrangement to acquire a preparation image of an acquisition region, the preparation image being defined with respect to a first coordinate system and used to define an examination image and at least one parameter for acquiring the examination image,
a storage interface configured to retrieve, from a storage including at least one prior imaging result data set of the subject, a source data set chosen from the at least one prior imaging result data set, each of the at least one prior imaging result data set including a prior image showing at least a part of the acquisition region and annotation information indicating at least one feature of interest of the subject, wherein the annotation information includes location information describing a location of a respective feature of interest in a second coordinate system of the prior image,
a registration unit configured to register the prior image and the preparation image to obtain registration information including a transformation from the second coordinate system to the first coordinate system,
a compilation unit configured to compile an output image including the preparation image and at least one visual representation, which is positioned correctly according to the location information for at least one direction, of all features of interest within a display region including the acquisition region, and
an output unit configured to output the output image via the display device.

14. A non-transitory computer-readable storage medium storing computer-readable instructions that, when executed by a computer, cause the computer to perform the computer-implemented method of claim 1.

15. The computer-implemented method according to claim 7, wherein the two-dimensional reference image is determined by forward projection from the prior image.

16. The computer-implemented method according to claim 15, wherein the two-dimensional reference image is determined by forward projection from the prior image based on an assumed acquisition geometry of the preparation image in the second coordinate system.

17. The computer-implemented method according to claim 10, wherein the at least one acquisition parameter for the examination image is an axial examination range.

18. The computer-implemented method according to claim 11, wherein the at least one direction includes a superior-inferior direction.

19. The computer-implemented method according to claim 12, wherein
the reference is to a slice,
the at least one extension of the feature includes a lesion diameter, and
the segmentation result is a three-dimensional segmentation result.

20. The medical imaging device of claim 13, wherein the medical imaging device is a computed tomography device.
